(19) United States
(12) Patent Application Publication
(10) Pub. No.: US 2009/0095306 A1
(43) Pub. Date: Apr. 16, 2009

(54) ORAL DEVICE FOR INTUBATION

(76) Inventors: Jeff Mandel, Media, PA (US); Marcos Amaro, Philidelphia, PA (US)

Correspondence Address: Pearl Cohen Zedek Latzer, LLP, 1500 Broadway, 12th Floor, New York, NY, 10036, US

(21) Appl. No.: 12/247,938

(22) Filed: Oct. 8, 2008

Related U.S. Application Data
(60) Provisional application No. 60/960,651, filed on Oct. 9, 2007.

(51) Int. Cl. A61B 1/00; A61M 16/00

(52) U.S. Cl. 128/845; 128/204.18

(57) ABSTRACT

This invention relates to a device for delivering positive airways pressure while effecting mandibular advancement and methods and systems comprising the same.

### CROSS REFERENCE TO RELATED APPLICATIONS

This application claims priority from U.S. Provisional Patent Application No. 60/960,651, filed 9 Oct., 2007, which is incorporated herein by reference in its entirety.

### FIELD OF INVENTION

This invention is directed to a device for providing positive airways pressure while maintaining an adjustable mandibular advancement, and methods and systems using the same.

### BACKGROUND OF THE INVENTION

Upper G1 endoscopy is a procedure commonly employed for a range of applications. The number of endoscopies is estimated at 2 5 million per year in the US. In Western populations, approximately 5% of patients experience heartburn on a daily basis. One third of patients undergoing endoscopy for evaluation of gastroesophageal reflux disease (GERD) have demonstrable erosive esophagitis, a known risk factor for development of esophageal cancer. Esophageal cancer is responsible for loss of 13,300 lives per year.

Routine screening for all patients with GERD for esophageal cancer may not be justifiable from a public health standpoint as the rate of complications of endoscopy may exceed the improvement in survival due to early detection. Upper GI endoscopy may be unpleasant, and many patients require sedation to tolerate the procedure. Complications attributable to sedation exceed technical complications by a factor of 10, chief amongst these are respiratory obstruction. The ability to utilize sedation during upper G1 endoscopy with reduced rates of respiratory complications would have a significant impact on an important public health problem.

Sedatives are known to cause upper airway collapse in dose-dependent fashion. During endoscopy, patients are sedated and a mouth gag inserted to facilitate the procedure, Mouth opening has been demonstrated to increase the potential for airway collapse in seated patients, mouth opening and head flexion exacerbates collapse of the passive airway. Upper airway collapse during sedation is in many ways similar to obstructive sleep apnea (OSA), In patients with OSA, obstruction of the upper airway is a frequent precursor to oxygen desaturation. The combination of sedation, mouth opening, and head flexion is a recipe for obstruction and desaturation during endoscopy.

Measures that have known efficacy in the management of OSA include continuous positive airway pressure (CPAP) and mandibular advancement. Application of CPAP has been demonstrated to decrease airway obstruction during midazolam sedation. Pressure support ventilation (PSV) via an endoscopy mask has been shown to reduce airway obstruction during propofol sedation. Mandibular advancement has shown efficacy in reducing airway obstruction during midazolam sedation. An oral appliance which provides mandibular advancement and allows application of CPAP or PSV during endoscopy could be expected to reduce the incidence of significant desaturation during endoscopy.

### SUMMARY OF THE INVENTION

In one embodiment, the invention provides a device for delivery of positive airway pressure and mandibular advancement comprising: a mandibular interface; a maxilar interface; and a midsection disposed therebetween, the midsection comprising an intubation port and a connection port capable of being coupled to a device for delivering positive airway pressure, and wherein the vertical alignment of the mandibular interface relative to the maxilar interface effects a mandibular advancement.

In another embodiment, the invention provides a method of conducting an upper GI endoscopy in a sedated subject, comprising the steps of: sedating the subject; inserting to the mouth of the subject a device for delivery of positive airway pressure and mandibular advancement, the device comprising: a mandibular interface; a maxilar interface; and a midsection disposed therebetween, the midsection comprises an intubation port and a connection port capable of being coupled to a device for delivering positive airway pressure, and wherein the vertical alignment of the mandibular interface relative to the maxilar interface effects mandibular advancement; attaching the connection port to a device for delivering positive airway pressure; and inserting a diagnostic endoscopy device into the intubation port.

In one embodiment, the invention provides a system for performing upper GI endoscopy comprising: a device for delivery of positive airway pressure and mandibular advancement comprising: a mandibular interface; a maxilar interface; and a midsection disposed therebetween, the midsection comprising an intubation port and a connection port capable of being coupled to a device for delivering positive airway pressure, and wherein the vertical alignment of the mandibular interface relative to the maxilar interface effects a mandibular advancement; a diagnostic endoscopy device; and a device for delivering positive airway pressure.

Other features and advantages of the present invention will become apparent from the following detailed description and figures. It should be understood, however, that the detailed description and figures, while indicating preferred embodiments of the invention are given by way of illustration only, since various changes and modifications within the spirit and scope of the invention will become apparent to those skilled in the art from this detailed description.

### BRIEF DESCRIPTION OF THE DRAWINGS

The invention will be better understood from a reading of the following detailed description taken in conjunction with the drawings in which like reference designators are used to designate like elements, and in which:

FIG. 1 shows an oral device (100) for providing positive airways pressure while maintaining mandibular advancement, the device comprising a mandibular interface (200), a maxillar interface (300), both arcuate in shape and substantially conforming to a lower and upper curvature of a teeth line of a subject, the device further comprising a midsection (400) giving two openings therein; an intubation port (401) and a connection port (402) capable of operably coupling to a device for maintaining positive airway pressure;

FIG. 2 shows a top and side view of the oral device (100); and a view of an embodiment of the intubation tube (401) further including a diaphragm to maintain the mask airtight.

FIG. 3 shows a side view of the lower skull with the lower jaw advanced and the relationship between the upper incisors 16 and the lower incisors 18. In performing mandibular advancement treatment, it is desired to advance in one embodiment, a lower jaw 10 to a position relative to the upper jaw 12.

### DETAILED DESCRIPTION OF THE INVENTION

This invention relates in one embodiment to a device for providing positive airways pressure while maintaining mandibular advancement. In other embodiments, the invention relates to methods or systems using the devices described herein.

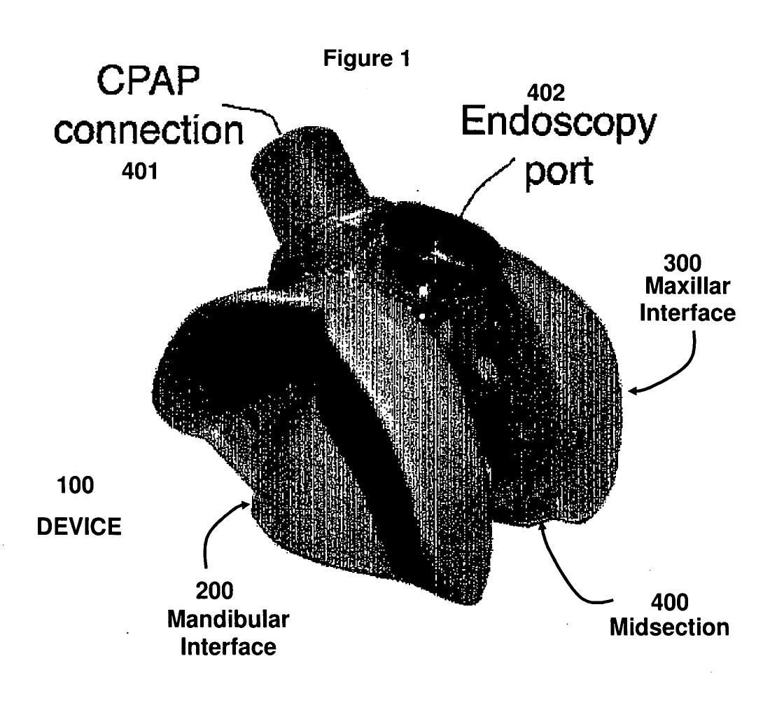

Referring now to the drawings; FIG. 1 illustrates an embodiment of a device (100) for delivery of positive airway pressure and mandibular advancement. The device (100) is comprised of a mandibular interface (200); a maxilar interface (300); and a midsection disposed therebetween (400), the midsection (400) comprising an intubation port (401) and a connection port (402), capable of being coupled to a device for delivering positive airway pressure. In an embodiment of the invention the vertical alignment of the mandibular interface (200) relative to the maxilar interface (300) effects a mandibular advancement when inserted to the subject mouth. In another embodiment, the mandibular advancement effected by the device (100) creates a horizontal gap of between 0 and 8 mm between the mandibular teeth line of the subject and the maxillar teeth line of the subject.

In another embodiment, the mandibular interface (200) and maxillar interface (300) are designed to substantially conform to the lower and upper teeth line respectively of a subject. In an embodiment of the device 100, the device for delivering positive airway pressure, connected to the connection port 402 is a 14 mm standard connector for a continuous positive airway pressure device (CPAP), or pressure support ventilation device (PSV) in another embodiment. In one embodiment, the intubation port 401 comprises a diaphragm adapted to allow the insertion of an intubation device therethrough, while maintaining a pressure seal in the oral cavity.

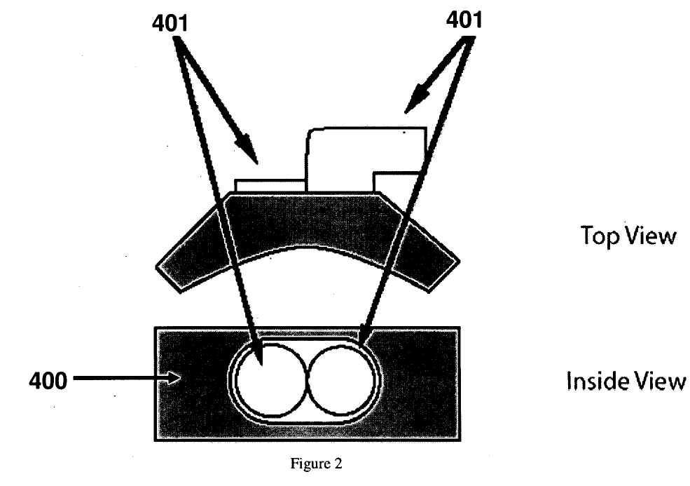

Referring now to FIG. 2, showing an embodiment of the midsection (400) from a top view and a side view, wherein the connection for adaptable for CPAP (401) and another connection port (402) are shown. The right or left configuration of the ports (401, 402) are by way of illustration only and may be interchanged according to other embodiments. Likewise the (inside) view of the midsection (400) shown as a rectangle, is by way of illustration only and may be any shape accommodating the maxillar (300) and mandibular (200) interfaces. The materials from which the device (100) is generally made from a colored molded plastic which is sufficiently soft to be cut for individual adaptation. It is contemplated that the device 100 will be made in several sizes to accommodate different sized jaws and different degrees of mandibular advancement. In one embodiment, the devices described herein, used as part of the methods and systems described herein, is comprised of mandibular interface and maxillar interface, which is comprised of olefins in one embodiment, or ethylene-vinyl acetate copolymer, ethylene-vinyl alcohol copolymer, polycaprolactone, polyvinyl chloride, polyesters, polycarbonates, polyamides, polyurethanes, polyesteramides, or their combination in other embodiments

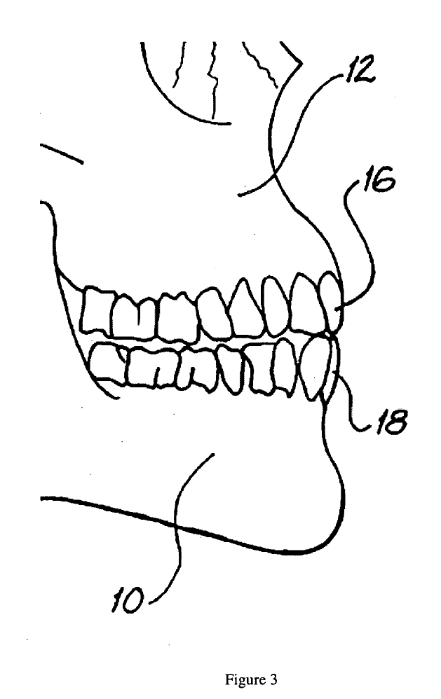

Referring now to FIG. 3, showing an embodiment of the relative position of the maxila 12 and mandible 10 from which the mandibular advancement of lower incisors 18 will be advanced using the devices described herein, over the upper incisors 16.

Accordingly and in one embodiment, provided herein is a device for delivery of positive airway pressure and mandibular advancement comprising: a mandibular interface; a maxilar interface; and a midsection disposed therebetween, the midsection comprising an intubation port and a connection port capable of being coupled to a device for delivering positive airway pressure, and wherein the vertical alignment of the mandibular interface relative to the maxilar interface effects a mandibular advancement. In another embodiment, the device for delivering positive airway pressure attached to the CPAP port 401, is a continuous positive airway pressure device (CPAP), or pressure support ventilation device (PSV). In another embodiment, the intubation port 402 is a diagnostic endoscopy port, or a feeding tube port, or a therapeutic agent delivery tube in other embodiments.

In one embodiment, the diagnostic endoscopy port 402, is operably linked to a bronchoscopy ventilation elbow.

referring now to FIG. 3 and in one embodiment, the devices provided herein affect mandibular advancement of between about 0 to about 10 mm of the lower incisors 18, relative to the upper incisors 16.

In one embodiment, the devices described herein, are used in the methods and systems described herein. Accordingly and in another embodiment, provided herein is a method of conducting an upper GI endoscopy in a sedated subject, comprising the steps of: sedating the subject; inserting to the mouth of the subject a device 100 for delivery of positive airway pressure and mandibular advancement, the device comprising: a mandibular interface 200; a maxilar interface 300; and a midsection 400 disposed therebetween, the midsection comprises an intubation port 402 and a connection port 401 capable of being coupled to a device for delivering positive airway pressure, and wherein the vertical alignment of the mandibular interface 200 relative to the maxilar interface 300 effects mandibular advancement; attaching the connection port 401 to a device for delivering positive airway pressure; and inserting a diagnostic endoscopy device into the intubation port 402.

In one embodiment, the midsection 400, further comprises an upper plate 201 operably coupled to the maxilar interface 200 and a lower plate 301 operably coupled to the mandibular interface 300, the upper and lower plates being slidably coupled and wherein the upper and lower plates are capable of being fixed in place. In another embodiment, the plates are adjusted to effect mandibular advancement of between about 2 and about 18 mm compared to a normal maxillary position relative to the mandible in a subject that is fully awake, prior to intubation or GI endoscopy performed using the methods described herein.

Likewise and in another embodiment, provided herein is a system for performing upper GI endoscopy comprising: a device 100 for delivery of positive airway pressure and mandibular advancement comprising: a mandibular interface 200; a maxilar interface 300; and a midsection 400 disposed therebetween, the midsection comprising an intubation port 402 and a connection port 401 capable of being coupled to a device for delivering positive airway pressure, and wherein the vertical alignment of the mandibular interface relative to the maxilar interface effects a mandibular advancement; a diagnostic endoscopy device; and a device for delivering positive airway pressure.

In one embodiment, the device for delivering positive airway pressure used in the methods and systems described herein, which is connected to the CPAP port 401, is a continuous positive airway pressure device (CPAP), or pressure support ventilation device (PSV). In another embodiment, the diagnostic endoscopy port 402 is operably linked to a bronchoscopy ventilation elbow is used in the methods and systems described herein. In one embodiment, the mandibular advancement effected using the device 100 described herein, by the relative position of the maxilar interface 300 and mandibular interface 200 is between about 0 to 10 mm as shown in the position of the upper incisors 16 relative to the lower incisors 18.

In one embodiment, using device 100 for delivery of positive airway pressure and mandibular advancement, the device comprising: a mandibular interface 200; a maxilar interface 300; and a midsection 400 disposed therebetween, the midsection comprises an intubation port 402 and a connection port 401 capable of being coupled to a device for delivering positive airway pressure, and wherein the vertical alignment of the mandibular interface 200 relative to the maxilar interface 300 effects mandibular advancement, which in one embodiment is between about 3 and 18 mm from normal wakefull state of the subject. In another embodiment, the vertical alignment of the mandibular interface 200 relative to the maxilar interface 300 effects mandibular advancement of between about 3 and 5 mm, or in another embodiment between about 5 and 8 mm, or in another embodiment between about 8 and 12 mm, or in another embodiment between about 12 and 15 mm, or in another embodiment between about 15 and 18 mm from normal wakefull state of the subject, each a discrete embodiment of the device provided herein.

In one embodiment, provided herein is a method of conducting an upper GI endoscopy in a sedated subject, comprising the steps of: sedating the subject; inserting to the mouth of the subject a device for delivery of positive airway pressure and mandibular advancement, the device comprising: a mandibular interface; a maxilar interface; and a midsection disposed therebetween, the midsection comprises an intubation port and a connection port capable of being coupled to a device for delivering positive airway pressure, and wherein the vertical alignment of the mandibular interface relative to the maxilar interface effects mandibular advancement; attaching the connection port to a device for delivering positive airway pressure; and inserting a diagnostic endoscopy device into the intubation port.

In another embodiment, the method of conducting an upper GI endoscopy is carried out in a non-sedated subject, and comprises the steps of; inserting to the mouth of the subject a device for delivery of positive airway pressure and mandibular advancement, the device comprising: a mandibular interface; a maxilar interface; and a midsection disposed therebetween, the midsection comprising an upper plate 201 operably coupled to the maxilar interface 200 and a lower plate 301 operably coupled to the mandibular interface 300, the upper and lower plates being slidably coupled and wherein the upper and lower plates are capable of being fixed in place; an intubation port; and a connection port capable of being coupled to a device for delivering positive airway pressure, using the slidably coupled plates, adjusting the mandibular advancement to between about 2 and 18 mm; attaching the connection port to a device for delivering positive airway pressure; and inserting a diagnostic endoscopy device into the intubation port.

In one embodiment, using the mandibular advancement device described herein results in an increase in pharengeal airways diameter as compared to a CPAP mask without a mandibular advancement. In another embodiment, provided herein is a method of preventing prevent upper airway collapse during sedation for an assessment of gastroesophageal reflux disease (GERD) in a subject, comprising the steps of inserting to the mouth of the subject a device for delivery of positive airway pressure and mandibular advancement, the device comprising: a mandibular interface; a maxilar interface; and a midsection disposed therebetween, the midsection comprising an upper plate 201 operably coupled to the maxilar interface 200 and a lower plate 301 operably coupled to the mandibular interface 300, the upper and lower plates being slidably coupled and wherein the upper and lower plates are capable of being fixed in place; an intubation port; and a connection port capable of being coupled to a device for delivering positive airway pressure, using the slidably coupled plates, adjusting the mandibular advancement to between about 2 and 18 mm; attaching the connection port to a device for delivering positive airway pressure; initiating continuous positive airway pressure and inserting a diagnostic endoscopy device into the intubation port, thereby maintaining airway patency.

In certain embodiments, the midsection will further comprise an additional connection port for attaching a device capable of continuously delivering an anesthetic gas.

Having described preferred embodiments of the invention with reference to the accompanying drawings, it is to be understood that the invention is not limited to the precise embodiments, and that various changes and modifications may be effected therein by those skilled in the art without departing from the scope or spirit of the invention as defined in the appended claims.

What is claimed is:

1. A device for delivery of positive airway pressure and mandibular advancement comprising:
a. a mandibular interface;
b. a maxilar interface; and
c. a midsection disposed therebetween, the midsection comprising an intubation port and a connection port capable of being coupled to a device for delivering positive airway pressure,
wherein the vertical alignment of the mandibular interface relative to the maxilar interface effects a mandibular advancement.

2. The device of claim 1, wherein the midsection further comprises an upper plate operably coupled to the maxilar interface and a lower plate operably coupled to the mandibular interface, the upper and lower plates being slidably coupled and wherein the upper and lower plates are capable of being fixed in place.

3. The device of claim 1, wherein the device for delivering positive airway pressure is a continuous positive airway pressure device (CPAP), or pressure support ventilation device (PSV).

4. The device of claim 1, wherein the intubation port is a diagnostic endoscopy port, a feeding tube port, or a therapeutic agent delivery tube.

5. The device of claim 4, wherein the diagnostic endoscopy port is operably linked to a bronchoscopy ventilation elbow.

6. The device of claim 1, wherein the mandibular interface is comprised of olefins, ethylene-vinyl acetate copolymer, ethylene-vinyl alcohol copolymer, polycaprolactone, polyvinyl chloride, polyesters, polycarbonates, polyamides, polyurethanes, polyesteramides, or their combination.

7. The device of claim 1, wherein the maxilar interface is comprised of olefins, ethylene-vinyl acetate copolymer, ethylene-vinyl alcohol copolymer, polycaprolactone, polyvinyl chloride, polyesters, polycarbonates, polyamides, polyurethanes, polyesteramides, or their combination.

8. The device of claim 1 or 2, wherein the mandibular advancement is between about 2 and 18 mm

9. A method of conducting an upper GI endoscopy in a sedated subject, comprising the steps of:
a. sedating the subject;
b. inserting to the mouth of the subject a device for delivery of positive airway pressure and mandibular advancement, the device comprising:
i. a mandibular interface;
ii. a maxilar interface; and
iii. a midsection disposed therebetween, the midsection comprises an intubation port and a connection port capable of being coupled to a device for delivering positive airway pressure, and wherein the vertical alignment of the mandibular interface relative to the maxilar interface effects a mandibular advancement;
c. attaching the connection port to a device for delivering positive airway pressure; and
d. inserting a diagnostic endoscopy device into the intubation port.

10. The method of claim 9, whereby the device for delivering positive airway pressure is a continuous positive airway pressure device (CPAP), or pressure support ventilation device (PSV).

11. The method of claim 9, whereby the diagnostic endoscopy port is operably linked to a bronchoscopy ventilation elbow.

12. The method of claim 9, whereby the mandibular advancement is between about 2 and 18 mm.

13. The method of claim 9, whereby the midsection further comprises an upper plate operably coupled to the maxilar interface and a lower plate operably coupled to the mandibular interface, the upper and lower plates being slidably coupled and wherein the upper and lower plates are capable of being fixed in place.

14. The method of claim 13, further comprising the step of adjusting the mandibular advancement prior to the step of inserting a diagnostic endoscopy device into the intubation port.

15. The method of claim 14, whereby the mandibular advancement is between about 2 and 18 mm.

16. A system for performing upper GI endoscopy comprising:
a. a device for delivery of positive airway pressure and mandibular advancement comprising:
i. a mandibular interface;
ii. a maxilar interface; and
iii. a midsection disposed therebetween, the midsection comprising an intubation port and a connection port capable of being coupled to a device for delivering positive airway pressure,
and wherein the vertical alignment of the mandibular interface relative to the maxilar interface effects a mandibular advancement;
b. a diagnostic endoscopy device; and
c. a device for delivering positive airway pressure.

17. The system of claim 16, wherein the device for delivering positive airway pressure is a continuous positive airway pressure device (CPAP), or pressure support ventilation device (PSV).

18. The system of claim 16, wherein the diagnostic endoscopy port is operably linked to a bronchoscopy ventilation elbow.

19. The system of claim 16, wherein the mandibular interface is comprised of olefins, ethylene-vinyl acetate copolymer, ethylene-vinyl alcohol copolymer, polycaprolactone, polyvinyl chloride, polyesters, polycarbonates, polyamides, polyurethanes, polyesteramides, or their combination.

20. The system of claim 16, wherein the maxilar interface is comprised of olefins, ethylene-vinyl acetate copolymer, ethylene-vinyl alcohol copolymer, polycaprolactone, polyvinyl chloride, polyesters, polycarbonates, polyamides, polyurethanes, polyesteramides, or their combination.

21. The system of claim 16, wherein the mandibular advancement is between about 2 and 18 mm.

22. The system of claim 16, wherein the midsection further comprises an upper plate operably coupled to the maxilar interface and a lower plate operably coupled to the mandibular interface, the upper and lower plates being slidably coupled and wherein the upper and lower plates are capable of being fixed in place.
